(19) United States
(12) Patent Application Publication
(10) Pub. No.: US 2006/0235329 A1
(43) Pub. Date: Oct. 19, 2006

(54) Reflex hammer

(76) Inventor: Dilip Bhavnani, Beverly Hills, CA (US)

Correspondence Address: THE SONI LAW FIRM, 55 S. LAKE AVE SUITE 720, PASADENA, CA, 91101, US

(21) Appl. No.: 11/107,454

(22) Filed: Apr. 14, 2005

(51) Int. Cl. A61B 19/00

(52) U.S. Cl. 600/553

(57) ABSTRACT

A folding reflex hammer includes two pivotally connected legs, one of which has the reflex testing head mounted on its inwardly facing side. The hammer is well suited to being carried by a physician or technician in any pocket due to its relatively small size when folded. In further embodiments, the folding reflex hammer may include the reflex testing device on one folding leg while the second folding leg may include additional functional implements, such as a writing implement. Other secondary implements, such as a neurological probe, may further be located within or may extend outwardly from the reflex testing head leg end. The hammer, having numerous large and generally flat sides, may further include imprinted areas for the purposes of advertising or promotion.

### FIELD OF THE INVENTION

The present invention is directed toward the improvement of one of the most commonly used devices in the medical profession. More specifically, the present invention is directed to a reflex hammer designed to be foldable and thereby readily portable and accessible to the practitioner. The present invention is also directed toward a reflex hammer which may incorporate additional functional features arising from its integration of secondary implement tips, probes or the like.

### BACKGROUND OF THE INVENTION

Reflex hammers have long been used in the medical profession. During most examinations, the physician, nurse or technician reaches for a reflex hammer to test the patients reflexes as a simple health or illness evaluation tool. The basic reflex hammer has typically consisted of only a flexible knob testing head attached to an elongated handle, which is swung like a hammer, as implied by the name. In its essence, the reflex hammer has changed little from its inception hundreds of years ago.

U.S. Pat. No. 4,324,261 to Mark, et al., describes a reflex hammer with a flexible handle. This design is but a minor improvement over the conventional steel handled implement and remains a cumbersome and unwieldy device. While the handle flexibility may improve use, the item is still of an obtrusive size and fit only for a singular purpose.

Similarly, U.S. Pat. No. 4,865,045, to Monreal describes a reflex hammer which incorporates a shoehorn on the device end opposite the hammer. Such a design only marginally improves the functionality, as it would be the patient, rather than the medical provider, who typically might require a shoehorn in an examination room. This only serves to increase the chances of the patient inadvertently leaving the office with the device still in their possession, causing the physician to search for a replacement device upon arrival of a subsequent patient. Moreover, this invention does nothing to overcome the unwieldy size or shape of the reflex hammer.

U.S. Pat. No. 5,657,763, to Schneider details a device which is designed to convert the hammer impact to an electrical signal. While perhaps an improvement upon the most basic design, this device is lacking, however, as it further contributes to the increased size and weight of the hammer. Moreover, the power source for the device is subject to limited battery life, which raises the specter of an inoperative instrument in the examination room.

Finally, U.S. Pat. No. 6,510,918, to Bates attempts to describe a combination device. That invention attempts to combine a stethoscope with a reflex hammer. Such a combination, however, results in a particularly gangly device, due to the extreme difference between the configuration of a stethoscope, with its extended tubes and earpieces, on the one hand, and the reflex hammer, on the other hand, with its basic construction of a handle and a striking end. This combination also requires partial disassembly of the device in order to achieve the multiple functions. Such construction would be destined to create problems for the user if any of the disassembled components become lost, broken or misplaced.

What is lacking in the field is a reflex hammer which is easily and unobtrusively carried by the provider so as to remain at the ready at all times. None of the presently existing devices in the field has attempted to reduce the size or to increase the portability of the device.

What is further needed is a multi function device which incorporates the hammer function along with one or more other useful apparatus. While some inventions have been introduced to promote additional functions as incorporated with the basic hammer, do date these inventions have done little to render a practical device. Instead, these devices have created even more unwieldy products through the incorporation of unnecessary, dissimilar or unwanted secondary functions. Those devices which incorporate a secondary function of interest to a medical provider have done so only at the sacrifice of practicality, compactness and usefulness of the resulting device.

Lastly, what is needed in a reflex hammer device is the ability to quickly and easily provide a variable stiffness and sensitivity hammer. While some devices presently known may incorporate this function, in order to do so these devices require additional bulky structure, such as electrically powered signal generation or conversion mechanisms.

### SUMMARY OF THE INVENTION

An object of the present invention is to provide a reflex hammer which will become a highly desirable and indeed, an indispensable item for the medical practitioner. To do so, the present invention is configured as a foldable device which the practitioner may easily and comfortably fit in any pocket.

A further object of the invention will be to provide the practitioner with a reflex hammer that serves many different yet essential purposes. For example, the contemplated reflex hammer device may further include a flashlight or a writing implement as an integral component within or extending from its structure. Similarly, a neurological probe or similar device may extend from and retract into one of the folding legs of the reflex hammer. As such, the invention imparts increased utility and purpose.

Another object of the invention is to provide a reflex hammer device which has an adjustable sensitivity control. This improvement will be especially important when the practitioner is examining the young or the elderly. Moreover, at times the instrument may be used to evaluate the reflex action of areas on the body which require more delicate touch than those commonly tested.

These and other objectives are achieved in the present invention through the development and configuration of a folding reflex hammer with increased functional capabilities in comparison to conventional devices for this task. The reflex hammer is comprised of two pivotally connected folding legs. The first such leg incorporates the typically triangular shaped striking device which contacts the specific portion of the patient's body. The second leg serves to form the grasping portion of the handle, as it would typically be fully open and extended in use.

This second leg, however, is not restricted to a mere opening handle function. In order to increase the functionality of the device, this portion of the hammer is a most appropriate location for the storage and deployment of additional implements. For example, a writing implement or a flashlight could be located within this leg. Such an implement may be stationary or may extend or retract, as needed.

In a similar fashion, the first leg may house or otherwise incorporate a secondarily functional tip. An example of such an element would be a pointed neurological probe which extends from or may be retracted into an orifice on the outboard end of the first end of the invention. By increasing the functionality of the reflex hammer, the present invention improves both the desirability and corresponding marketability of the object.

The sensitivity adjustment is achieved through the use of a sliding lever on the handle which is affixed to a tensioning device, in turn attached to the striking hammer. In the case of the sensitivity adjustment embodiment, the striking head element would be an alternate construction, such as a hollow polymer or rubber type of structure. By increasing the tension on a tensioning device, such as a tensioning cord, the striking head is compressed, thereby increasing its stiffness. As the stiffness of the striking head is increased or decreased, so varies the sensitivity of the instrument.

### BRIEF DESCRIPTION OF THE DRAWINGS

These and other aspects of the invention will now be described with reference to the accompanying drawings, in which:

FIG. 1 is a perspective view of a preferred embodiment of the Reflex Hammer, which depicts the hammer in its fully open and extended position.

FIG. 2 is a perspective view of a preferred embodiment of the Reflex Hammer, which depicts hammer in a position where it is approximately one half open and closed.

FIG. 3 is a perspective view of a preferred embodiment of the Reflex Hammer, which depicts the hammer in its fully closed position

FIG. 4 is a perspective view of a preferred embodiment of the Reflex Hammer, which depicts the hammer in its fully open and extended position including an alternate option for the embodiment whereby a miniature flashlight is mounted in the handle base of the hammer.

FIG. 5 is a perspective view of a preferred embodiment of the Reflex Hammer, which depicts the hammer in its fully open and extended position including an alternate option for the embodiment whereby a writing implement is mounted in the handle base of the hammer.

FIG. 6 is a perspective view of a preferred embodiment of the Reflex Hammer, which depicts the hammer in its fully open and extended position including an alternate sensitivity mechanism which is actuated by a slide member attached to a tensioning device. In this drawing, the tensioning slide member is in its lowest position, which corresponds to the most stiff and lowest sensitivity striking head.

FIG. 7 is a side view of a preferred embodiment of the Reflex Hammer, which depicts the hammer in its fully open and extended position including an alternate sensitivity mechanism which is actuated by a slide member attached to a tensioning device. In this drawing, the tensioning slide member is in its lowest position, which corresponds to the most stiff and lowest sensitivity striking head.

FIG. 8 is a perspective view of a preferred embodiment of the Reflex Hammer, which depicts the hammer in its fully open and extended position including an alternate mechanism which is actuated by a slide member attached to an extending probe device. In this drawing, the slide member is in its lowest position, which corresponds to the retraction of the probe into the elongated leg of the striking end.

FIG. 9 is a side view of a preferred embodiment of the Reflex Hammer, which depicts the hammer in its fully open and extended position including an alternate mechanism which is actuated by a slide member attached to an extending probe device. In this drawing, the slide member is in its lowest position, which corresponds to the retraction of the probe into the elongated leg of the striking end.

FIG. 10 is a perspective view of a preferred embodiment of the Reflex Hammer, which depicts the hammer in its fully open and extended position including an alternate mechanism which is actuated by a slide member attached to an extending probe device. In this drawing, the slide member is in its highest position, whereby the probe is extended outwardly from the elongated leg of the striking end.

FIG. 11 is a side view of a preferred embodiment of the Reflex Hammer, which depicts the hammer in its fully open and extended position including an alternate mechanism which is actuated by a slide member attached to an extending probe device. In this drawing, the slide member is in its highest position, whereby the probe is extended outwardly from the elongated leg of the striking end.

### DETAILED DESCRIPTION OF THE INVENTION

The invention described herein comprises a folding instrument which is utilized by health care professionals for patient testing and diagnostic purposes. One of the most basic medical tests performed daily in examination rooms is the patient's reflex test typically at the knee, elbow or other similar neurological sites. To conduct the test, the physician strikes the patient sharply yet lightly, with an hammer that typically has a soft striking head. In order to render this reflex hammer more convenient, useful and desirable, the present invention takes the product from an unwieldy and cumbersome device and brings to fruition an updated, folding multi-functional device.

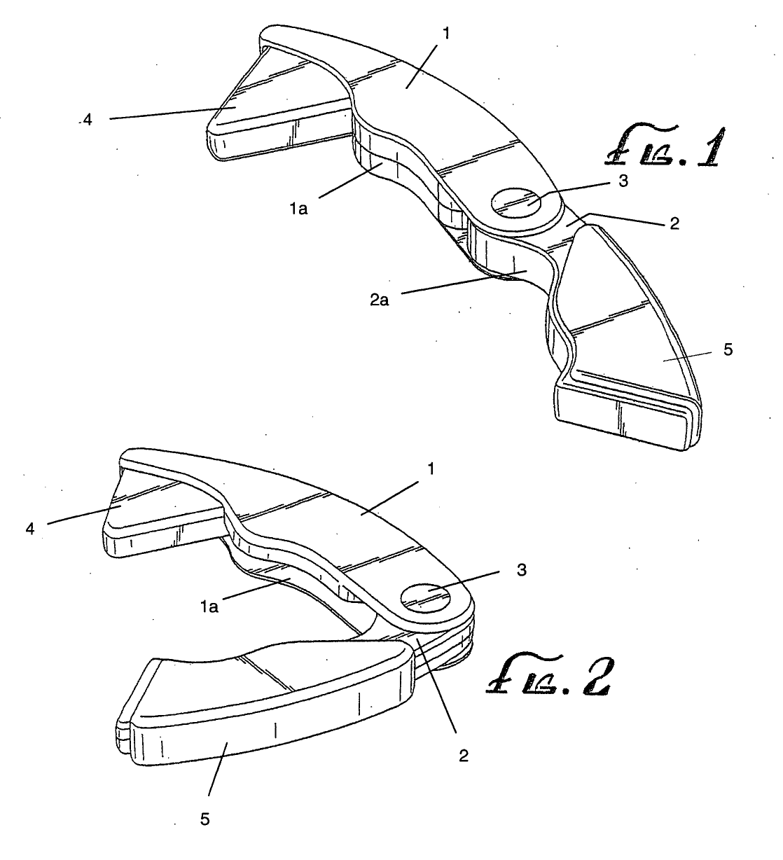

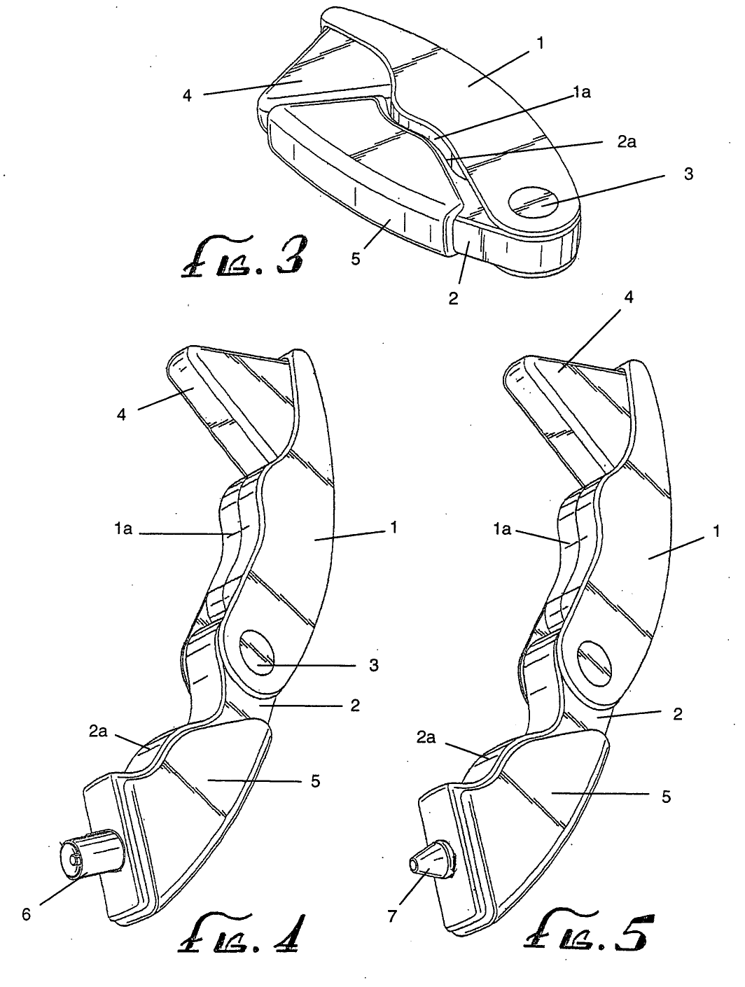

In a preferred embodiment, the folding hammer is essentially comprised of three parts. Referring to FIGS. 1-3, the preferred embodiment comprises a first elongated leg 1, and a second elongated leg 2, which are pivotally connected by a pivot pin 3. The pivot pin 3 permits the legs to fold in a fashion similar to that of a pocketknife. As seen in FIG. 3, when folded, the invention becomes very compact and is easily ported in the user's pocket. In use, the invention would be completely opened and the second leg 2 would become the bottom portion of the handle or hand grip, with the user's thumb and forefinger extending toward the lower portion of the first elongated leg 1, just above the pivot pin 3. In the preferred embodiment, as depicted in FIG. 1, the second leg 2 and the first leg 1 each have a contoured portion, 1a and 2a, respectively, such that the opened device readily conforms to the user's grasp. Moreover, these contoured portions 1a and 2a are configured so as to next within and adjacent to one another when the device is closed, as shown in FIG. 3, to minimize the folded size of the invention.

The first elongated end further includes a striking head 4, which would most typically be constructed of a rubber, polymer or plastic material. The second end 2 may optionally include a secondary gripping handle 5, which may be constructed of a variety of materials and would usually serve to improve the gripping surface of the hammer when extended.

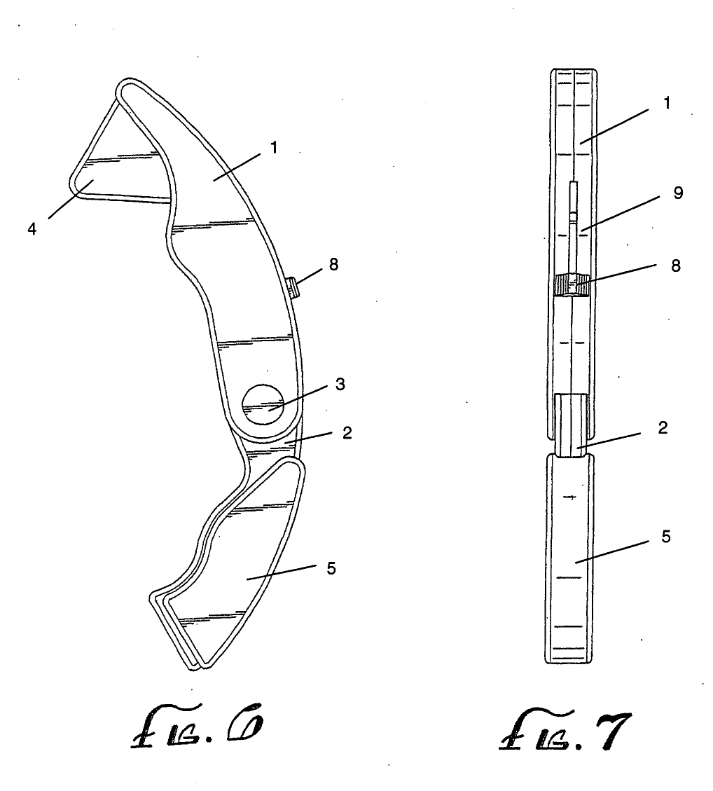

In a preferred embodiment, the invention may include only a folding reflex hammer, as generally described above. In order to increase the functional utility of the device, however, a host of optional implements may also be located on or displaced within the invention. For example, the second leg 2 may further include one or more implements such as those shown in FIGS. 4 and 5. In FIG. 4, an optional flashlight 6 is shown to be extending from the second leg 2. Such a device could be retractable into the leg, permanently extending from the leg as shown, or the device may flip out from the inner face at the contoured portion 2a, similar to multi-function tool devices well known in the art which have pliers, knives and other implements. The writing implement 7, as shown in FIG. 5, may also be permanently mounted on the surface or may be a retractable implement tip.

While these alternate embodiments are of a flashlight 6 and a pen 7, this invention is well suited to and applicable for any hand held implement to be incorporated in the second leg 2. Examples of such implements contemplated within the scope of the present invention include writing implements such a pens and pencils, laser pointers, conventional pointers, cutting implements such as knives, awls and scribes, and other hand-held implements such as brushes, cosmetics applicators, testing heads and computer styluses.

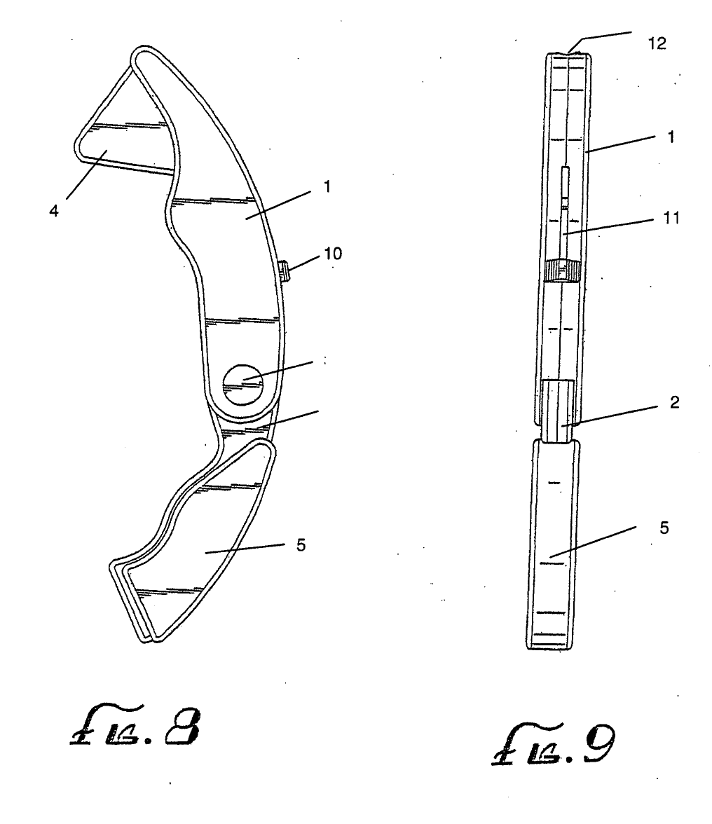

In some cases, a sensitivity or stiffness adjustment of the striking head might be desirable. This feature could be utilized when using the invention on an infant, on an elderly patient or if the health care provider wishes to utilize the device on an extremely sensitive part of the body. The present invention is well suited to an embodiment wherein the stiffness or sensitivity is configured to be adjustable. As shown in FIGS. 6 and 7, for example, one method to achieve adjustability would be to incorporate a tensioning chord attached to a tensioning slide 8 which slides in a slide track 9. The slide 8 could be held in position by a spring loaded cam mechanism which pinches the chord to maintain its position. Alternatively, a ratcheting mechanism could be used to hold the slide in position. The cam and ratcheting mechanisms are both well known to those skilled in the art. One end of the tensioning chord would be attached to the slide 8 with the second end attached to the inside of the striking head 5 at its striking tip. In this instance the striking head 5 would be hollow and therefore, compressible. By moving the slide 8 from its uppermost to its lowest position, the tension chord would be pulled tighter and correspondingly, the striking head 5 would be compressed. The more compressed the head becomes, the stiffer it becomes, in turn reducing its sensitivity. By moving the slide 8 up and down in the slide track 9, the provider could adjust the device to a position wherein they are most comfortable to sense a patient response or they could adjust the striking head 5 to become softer or firmer for a particular intended use.

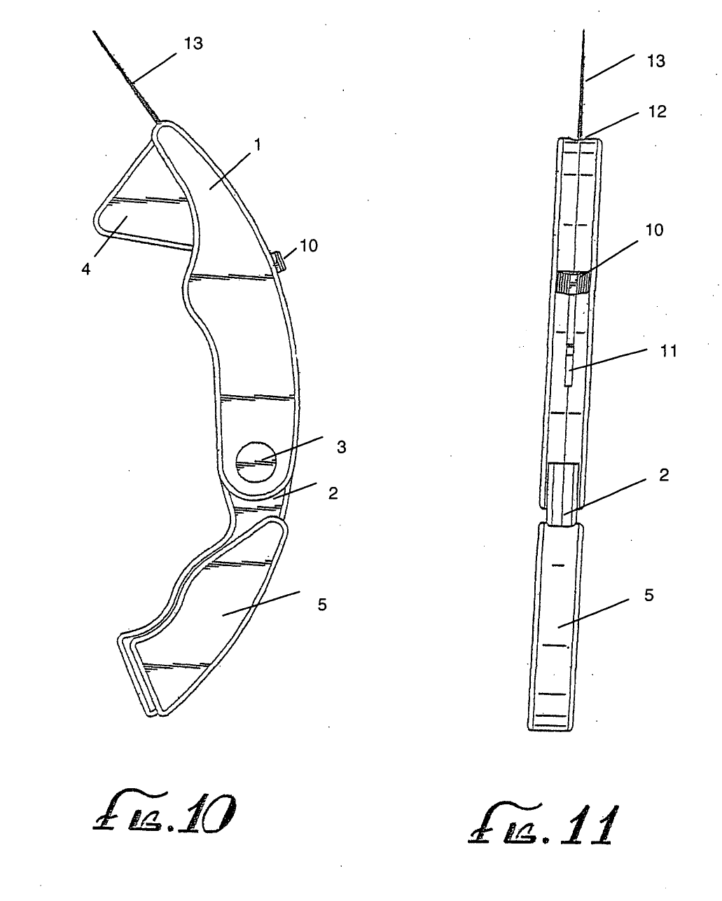

While the second end 2 of the invention is well suited for a secondary implement location, so too might an implement be located at the outboard end of the first end 1. One such implement would be a pointed sensing probe such as that used by a physician to test various areas of the skin and extremities for neurological response. FIGS. 8 through 11 depict a mechanism whereby a retractable probe 13 extends from and may be retracted into an orifice 12. The probe 13 could thereby be included as a secondarily functional tip of the hammer. In a most basic form, the innermost end of the probe 13 is affixed to an extender slide 10, which may slide up and down within a slide track 11. As shown in FIGS. 8 and 9, when the slide 10 is in its lowest position in the track 11, the probe 13 is retracted into the first end 1 of the hammer. Sliding the extender slide 10 toward its upper position extends the probe 13 outward through the orifice 12 in the first end 1, as in FIGS. 10 and 11.

The above descriptions are merely exemplar as to what types of secondary implements may be included with the folding reflex hammer. Any configuration of implement, tip probe or other functional device commonly utilized in the medical profession which may be adapted to fit the hammer configuration is contemplated to be within the scope of this invention.

Lastly, it is contemplated that any of the numerous generally flat surfaces of the folding reflex hammer are well suited for and may be utilized for the imprinting of graphical or advertising material for the purposes of advertising or promotion.

What is claimed is:

1. A folding reflex hammer having a first elongated leg and a second elongated leg;
said first leg further including a reflex testing striking head proximate one end and a pivot location on the opposite end of said leg; and
said second leg pivotally connected to the first leg so as to create a folding assembly.

2. The folding reflex hammer of claim 1, wherein the reflex testing striking head is generally triangular shaped.

3. The folding reflex hammer of claim 1, wherein the reflex testing striking head is constructed of a flexible material.

4. The folding reflex hammer of claim 1, wherein the reflex testing striking head is hollow.

5. The folding reflex hammer of claim 1, wherein the second elongated leg further includes an implement tip selected from the group including: a knife, a writing instrument, a flashlight, a laser pointer, a stylus point, a probe, a star wheel and a screwdriver.

6. The folding reflex hammer of claim 1, wherein advertising or graphical material is imprinted on at least one outer surface of the reflex hammer for the purpose of advertising or promotion.

7. The folding reflex hammer of claim 1, wherein the first elongated leg further includes an implement tip which may be retracted or extended through an orifice in said end.

8. The folding reflex hammer of claim 7, wherein the implement tip comprises a neurological probe.

9. The folding reflex hammer of claim 4, wherein one leg further includes a sensitivity adjustment mechanism.

10. The folding reflex hammer of claim 9, wherein the sensitivity adjustment mechanism comprises a sliding member which alternatively increases or decreases the stiffness of the striking head based on the position of a sliding member.

11. A method of advertising or promotion comprising the imprinting of printed or graphical material on a surface of a folding reflex hammer which has a first elongated leg and a second elongated leg; said first leg further including a flexible reflex testing striking head proximate one end and a pivot location on the opposite end of said leg; and said second leg pivotally connected to the first leg so as to create a folding assembly.
